(19) United States
(12) Patent Application Publication
(10) Pub. No.: US 2016/0355870 A1
(43) Pub. Date: Dec. 8, 2016

(54) GENERATION OF LIGATION-READY DNA AMPLICONS

(71) Applicant: QIAGEN GMBH, Hilden (DE)

(72) Inventors: Nan Fang, Neuss (DE); Dirk Loeffert, Hilden (DE)

(73) Assignee: QIAGEN GMBH, Hilden (DE)

(21) Appl. No.: 15/038,502

(22) PCT Filed: Oct. 13, 2014
(86) PCT No.: PCT/EP2014/071897

§ 371 Date: May 23, 2016

(30) Foreign Application Priority Data
Nov. 26, 2013 (EP) 13194453.0

(51) Int. Cl. C12Q 1/68; C12P 19/34

(52) U.S. Cl. CPC C12Q 1/686; C12P 19/34

(57) ABSTRACT

The invention is directed to novel methods, kits and uses to be employed for the generation of ligation-ready DNA amplicons of a target DNA by using 5′-phosphorylated primers.

The invention is directed to novel methods, kits and uses to be employed for the generation of ligation-ready DNA amplicons of a target DNA.

### FIELD OF THE INVENTION

The present invention relates to the field of molecular biology, more particularly to the generation of ligation-ready DNA amplicons and, specifically, to the generation of DNA-adaptor-ligated DNA amplicons of a target DNA, respectively.

### BACKGROUND OF THE INVENTION

In the field of molecular or recombinant biology ligation-ready DNA amplicons are required to generate DNA-adaptor-ligated DNA amplicons of a target DNA in order to subject the DNA amplicons to a subsequent processing, such as sequencing or amplification.

There are two common methods to generate amplicon libraries which can be used for platform-specific sequencing. One method uses conventional multi-step enzymatic reactions to ligate DNA adaptor molecules to the amplicons. The amplicons are generated with target-specific primers by PCR, and the amplification product is then end-repaired. The end-repair step usually requires two enzymes, a polynucleotide kinase, such as T4 TNK that phosphorylates the 5′-end of the double-stranded PCR product, and enzymes with polymerase and exonuclease activities that make the ends of the PCR products blunt by either fill-in or trimming, respectively. After the end-repair for sequencing on an Illumina® or similar platform an adenylation (A-addition) is required where an A-overhang is added to the 3′-end of end-repaired PCR product, usually by the Klenow exo-minus fragment. This is to generate a docking site for the sequencing adaptors that comprise a T-overhang. After A-addition, the sequencing adaptor can be ligated to the amplicon by a DNA ligase, usually the T4 DNA ligase; cf. Illumina® TruSeq™ DNA Sample Preparation v2 Guide. For other sequencing platforms the A-addition step is not needed and blunt-ended, 5′-phosphorylated adaptors are directly ligated to the end-repaired amplicons; cf. Life Technologies, Ion Xpress™ Plus gDNA and Amplicon library preparation.

Another method to reduce adaptor ligated DNA amplicons for sequencing is to use fusion PCR primers that contain both target-specific sequence and part of the adaptor sequences. After the first round of the PCR and the amplification of the target-specific regions, a second round of the PCR can be performed with PCR primers containing the complete adaptor sequence to add the adaptor sequence to the amplicon.

The WO 2007/037678 discloses a method of preparing a sequence library for high-throughput sequencing using 454 Life Science technology.

Beem et al. (2009), Lambda Chops: Creation of Site-Directed Mutants in Insertable Fragments Utilizing Gataeway® Technology, Mol. Biotechnol. 42, p. 275-281, describe a method to produce site-directed mutations within a cDNA by assembling mutagenized PCR fragments in proper orientation using lambda integration in an extension of Gateway technology.

Sathe and Koser (1992), Use of Phosphorylated Oligodeoxynucleotide Primers in the Cloning of Polymerase Chain Reaction Products, Methods in Molecular and Cellular Biology 3, p. 188-189, describe a method for PCR fragment cloning which utilizes chemically phosphorylated oligodeoxynucleotide primers in a polymerase chain reaction to generate a DNA fragment which can be inserted into a vector at a blunt and dephoshorylated restriction site.

The methods of the prior art are tedious and time-consuming. Moreover, the method using fusion PCR primers could also post challenge on the design of suitable PCR primers.

Against this background, it is an object of the present invention to provide a method for generating ligation-ready DNA amplicons of a target DNA where problems associated with the prior art methods can be reduced or avoided.

The present invention satisfies these and other needs.

### SUMMARY OF THE INVENTION

The present invention provides a method for generating ligation-ready DNA amplicons of a target DNA, comprising (i) contacting in a polymerase chain reaction (PCR) buffer said target DNA with at least one DNA polymerase, a dNTP mixture, and at least one PCR primer pair consisting of two target specific PCR primers, to obtain a reaction mixture,(ii) subjecting said reaction mixture to a PCR to generate a plurality of ligation-ready DNA amplicons of said target DNA, wherein at least one of said target specific PCR primers is 5′-phosphorylated.

The present invention also provides the use of a 5′-phosphorylated PCR primer for generating ligation-ready DNA amplicons of a target DNA.

The inventors have surprisingly realized that amplifying a target DNA by PCR under common conditions, however using at least one 5′-phosphorylated PCR primer, results in the generation of a plurality of ligation-ready DNA amplicons of said target DNA.

As used herein, “target DNA” refers to any single-stranded DNA (ssDNA) or double-stranded DNA (dsDNA) of interest of which the generation of ligation-ready DNA amplicons is intended. “Target DNA” can be derived from any in vivo or in vitro source, including from one or multiple cells, tissues, organs, or organisms, whether living or dead, whether prokaryotic or eukaryotic, or from any biological or environmental source. Typically but not exclusively, “target DNA” refers to such ssDNA or dsDNA the nucleotide sequence of which is to be elucidated by sequencing, e.g. next generation sequencing (NGS).

As used herein, “DNA amplicon” refers to a DNA molecule that is the source and/or product of amplification or replication events, e.g. formed by polymerase chain reactions (PCR). In this context, “amplification” refers to the production of one or more copies of a genetic fragment or target sequence, specifically the amplicon.

“Ligation-ready” as used herein refers to the state of the DNA amplicon allowing a direct ligation of the latter with another DNA molecule. “Another DNA molecule” could be a DNA adapter molecule, which may comprise a nucleotide sequence for annealing a PCR or sequencing primer. “Direct ligation” in this context means that no intermediate steps such as end repair, adenylation of 3′-ends (A-addition) or other intermediate enzymatic reactions are required before the DNA ligase reaction can successfully catalyze the joining of the “another DNA molecule” or DNA adaptor molecule to the DNA amplicon, respectively.

As used herein, a “PCR buffer” refers to such a buffer solution allowing the processes of the PCR and, thus, the generation of the DNA amplicons. An example for such a “PCR buffer” is the QIAGEN® PCR Buffer.

As used herein, “dNTP mixture” refers to a PCR conventional mixture of the deoxynucleoside triphosphates dATP, dGTP, dCTP, dTTP, i.e. the building-blocks from which the DNA polymerase synthesizes a new DNA strand.

A “DNA polymerase” as used herein, refers to such a DNA polymerase that functions under PCR conditions and includes thermo-stable or heat-stable DNA polymerases. “At least one” DNA polymerase in this context means that one, two, three or more or even a mixture of different DNA polymerases, respectively, can be used.

“Polymerase chain reaction” or “PCR” as used herein refers to a conventional polymerase chain reaction but includes all kinds of PCRs allowing the generation of DNA amplicons of a target DNA.

According to the invention, the “PCR primer pair” is consisting of two target-specific PCR primers. That means, each of the two target-specific PCR primers comprise a sequence allowing a specific hybridization to a section of the target DNA defining the starting point for the DNA synthesis. As this is perfectly known to the skilled person, the two target-specific PCR primers are preferably chosen as to limit on both sides of the target DNA a range to be replicated. “At least one” PCR primer pair means one, two, three or more or even a mixture of different PCR primer pairs can be used.

“5′-phosphorylated” in the context of the invention means that at least one of the target specific PCR primers comprises at its 5′-end one or more phosphate groups. The phosphate group(s) can be joined to the 5′-end enzymatically, e.g. by kinases, or via chemical synthesis, e.g. using chemical phosphorylation reagents. In this context, “at least one target-specific PCR primer” means that one or both target-specific PCR primers of the at least one PCR primer pair can be 5′-phosphorylated.

The object underlying the invention is herewith completely solved.

The method according to the invention is far from being obvious.

In the art it has been assumed that the generation of ligation-ready DNA amplicons of a target DNA compellingly requires complex enzymatic reactions. Therefore, it was surprising that the use of 5′-phosphorylated PCR primers in cooperation with the PCR polymerase to amplify a target DNA by a PCR results in a plurality of ligation-ready DNA amplicons of said a target DNA.

According to a further development of the method of the invention the DNA polymerase has no 3′-5′ exonuclease activity but terminal transferase activity, whereby it is preferred that said DNA polymerase is a Taq polymerase. The Taq polymerase has been proven as being particularly suited for this preferred embodiment.

This measure has the advantage that amplicons are provided with adenylate (A) overhangs which are e.g. required for Illumina® sequencing platforms.

According to a preferred alternative embodiment of the method according to the invention, said DNA polymerase has a 3′-5′ exonuclease activity, whereby it is preferred that said DNA polymerase is a Pfu or KOD polymerase.

This measure has the advantage that blunt-ended amplicons are generated which are suitable for various sequencing platforms. The polymerases Pfu or KOD have been proven as being particularly suited for realizing this embodiment.

According to a further development of the method of the invention, the ligation-ready DNA amplicons are configured for a ligation with a DNA adaptor molecule comprising a nucleotide sequence for annealing an oligonucleotide.

This measure has the advantage that the DNA amplicons are provided in such a state allowing the ligation with DNA molecules necessary for further processing such as sequencing or amplifying.

As used herein, “DNA adaptor molecule” refers to a single-stranded DNA (ssDNA) or double-stranded DNA (dsDNA) ligatable to the DNA amplicon, preferably configured for annealing an oligonucleotide such as a PCR or sequencing primer. The ligation of the DNA adaptor molecule to the DNA amplicon provides for a state of the latter “ready-for-sequencing” or “ready-for-amplifying”, respectively.

According to a preferred embodiment, the method of the invention is further comprising (iii) isolating said plurality of ligation-ready DNA amplicons of said a target DNA from said reaction mixture.

“Isolating” can be understood as purifying the DNA amplicons by removing the DNA polymerase, the remaining dNTPs, and the PCR buffer, respectively. Such measure has the advantage that the DNA amplicons are brought in a condition that allows their direct introduction into a subsequent reaction, such as a ligation reaction.

Another subject-matter of the present invention relates to method for generating DNA-adaptor-ligated DNA amplicons of a target DNA, comprising (i) contacting in a polymerase chain reaction (PCR) buffer said target DNA with at least one DNA polymerase, a dNTP mixture, and at least one PCR primer pair consisting of two target specific PCR primers, to obtain a reaction mixture, (ii) subjecting said reaction mixture to a PCR to generate a plurality of ligation-ready DNA amplicons of said target DNA, (iii) isolating said plurality of ligation-ready DNA amplicons of said target DNA from said reaction mixture, (iv) ligating said ligation-ready DNA amplicons of said target DNA to at least one DNA adaptor molecule to generate DNA-adaptor-ligated DNA amplicons of said target DNA, wherein at least one of said target specific PCR primers is 5′-phosphorylated.

The characteristics, features and advantages of the method for generating ligation-ready DNA amplicons of a target DNA also apply for such method for generating DNA-adaptor-ligated DNA amplicons of a target DNA. The latter method essentially differs from the first method by the ligating step (iv).

By the ligating step (iv), at least one DNA adaptor molecule is joined to the 3′- and/or 5′-end(s) of the single- or double-stranded DNA amplicons. Such ligation is preferably catalyzed by a DNA ligase, therefore, step (iv) may be performed under conditions where a DNA ligase can exert its function.

Regarding further developments of the DNA polymerase, it is referred to the method for generating ligation-ready DNA amplicons of a target DNA as set out above.

According to a further development, said DNA adaptor molecule comprises a sequence for annealing an oligonucleotide, which is preferably configured for annealing a PCR and/or sequencing primer, more preferred for annealing a primer for clonal amplification and/or for next generation sequencing (NGS).

This measure has the advantage that the DNA amplicons of the target DNA are provided in a state immediately usable in a subsequent amplification or sequencing reaction.

The generation of ligation-ready DNA amplicons or DNA-adaptor-ligated DNA amplicons of a target DNA also includes the concept of the generation of a library of ligation-ready DNA amplicons or DNA-adaptor-ligated DNA amplicons of a target DNA, respectively.

The methods according to the invention can be performed within one reaction vessel. This measure embodies the principle of a “one-step-method”. Even though the method according to the invention is subdivided into (i), (ii), and (iii), and eventually (iv), this subdivision only intends to illustrate the chronological sequence of the method events. In particular the method for generating DNA-adaptor-ligated DNA amplicons of a target DNA requires only one single ligation step and no commonly required end-repair and A-addition steps which are time-consuming and inefficient.

Another subject-matter of the present invention relates to a kit for generating ligation-ready DNA amplicons of a target DNA, comprising (i) at least one DNA polymerase, (ii) a dNTP mixture, and (iii) at least one 5′-phosphorylated target specific PCR primer.

Still another subject-matter of the present invention relates to a kit for generating DNA adaptor ligated DNA amplicons of a target DNA, comprising (i) at least one DNA polymerase, (ii) a dNTP mixture, (iii) at least one 5′-phosphorylated target specific PCR primer, and (iv) at least one DNA adaptor molecule.

A kit is a combination of individual elements useful for carrying out the methods of the invention, wherein the elements are optimized for use together in the methods. The kits also contain a manual for performing the respective method according to the invention. Such kits unify all essential elements required to work the methods according to the invention, thus minimizing the risk of errors. Therefore, such kits also allow semi-skilled laboratory staff to perform the methods according to the invention.

Unless defined otherwise, all technical and scientific terms used herein generally have the same meaning as commonly understood by one of ordinary skill in the art to which this invention pertains.

The characteristics, features and advantages of the methods according to the invention apply to the kits according to the invention correspondingly.

It goes without saying that the above-mentioned features and the features which are still to be explained below can be used not only in the respective specified combinations, but also in other combinations or on their own, without departing from the scope of the present invention.

Further features, characteristics and advantages follow from the description of preferred embodiments and the attached figure.

In the Figure:

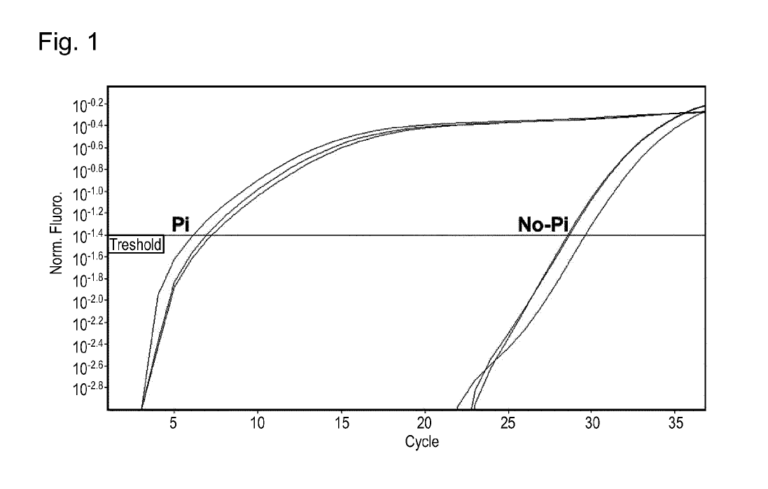

FIG. 1 shows a graph illustrating the increase of ligated IL1 R2 amplicons generated by the method according to the invention depending on the number of PCR cycles.

### EXAMPLES

To prove the principle of the invention, the inventors used the claimed method to perform a PCR using Taq polymerase and 5′-phosphorylated PCR primers. The inventors then directly ligated the amplicons to Illumina® TruSeq sequencing adapters which have a T-overhang.

Briefly, 50 μl PCR reactions were set up with 20 ng of human gemonic DNA as template, 0.2 mM of each of dNTPs, 1.25 U of QIAGEN Taq polymerase (5 U/μl), 1×QIAGEN® PCR Buffer, and 0.2 μM each of PCR primers (SEQ ID NO. 1 and SEQ ID NO. 2) that specifically recognize the human IL1 R2 gene. The IL1 R2 primers were either 5′-phosphorylated or unmodified. The PCR cycling conditions were as follows: 94° C., 5 minutes for denaturation; then 35 cycles of 94° C., 30 seconds; 60° C., 30 seconds; and 72° C., 30 seconds; followed by a 72° C., 10 minutes final extension during which the terminal transferase activity would result in the addition of one single A nucleotide at the 3′-ends of the PCR products. Once PCR was completed, the PCR reaction was cleaned up with MinElute PCR Purification kit (Qiagen®) and eluted with 28 μl RNase-free water.

25 μl of the purified PCR product or amplicon, respectively, was then subjected to a ligation reaction with 1×NEBNext® Quick Ligation Reaction Buffer (NEB), 2.5 μM of Illumina® adapter that was generated by annealing to oligos (IDT, SEQ ID NO. 3 and SEQ ID NO. 4) to form a duplex, 5 μl Quick T4 Ligase (NEB) in a 50 μl reaction for 15 minutes at 20° C.

After ligation, the reaction products were again purified with MinElute PCR purification kit (Qiagen®) and eluted with 50 μl EB buffer, and diluted with 1:100 RNase-free water. Quantitative, real-time PCR (qPCR) using primers recognizing Illumina® adapter sequences (SEQ ID NO. 5 and SEQ ID NO. 6) was then used to quantify the PCR products that had been ligated with the adaptors. A 25 μl qPCR reaction contained QuantiFast Sybr Green PCR Mix (1×), 1 μM each of the primers specifically recognizing Illumina® adapter sequences, and 1 μl diluted ligation product. qPCR cycling conditions were as follows: 95° C., 5 minutes; and 40 cycles of 95° C., 10 seconds; 60° C., 30 seconds.

As shown in FIG. 1 and Table 1, the ligation product generated from amplicons with 5′-phosphorylated PCR primers (Pi, Ct mean of 6.74) could be detected with much lower Ct-values than the ligation product generated from amplicons with non-phosphorylated PCR primers (No-Pi, Ct mean of 28.94). The Ct value reflects the quantity of the generated DNA. The lower the Ct-value, the more DNA has been generated.

TABLE 1
Ct values of the ligated amplicons that can be detected
with PCR primers recognizing adaptor sequences

| Name | Ct | Ct, Mean |
|---|---|---|
| Pi | 7.24 | 6.74 |
| Pi | 6.89 | |
| Pi | 6.10 | |
| No-Pi | 28.65 | 28.94 |
| No-Pi | 28.56 | |
| No-Pi | 29.62 | |

Sequences:

SEQ ID NO. 1:
5′-cgg gta ggc gct ctc tat gt-3′
SEQ ID NO. 2:
5′-aag act gac aat ccc gtg taa gg-3′
SEQ ID NO. 3:
AATGATACGGCGACCACCGAGATCTACACTCTTTCCCTACAC
GACGCTCTTCCGATC*T (*: indicates
phosphorothioate)
SEQ ID NO. 4:
GATCGGAAGAGCGGTTCAGCAGGAATGCCGAGACCGATCTC
GTATGCCGTCTTCTGCTT* (*: indicates
phosphorothioate)
SEQ ID NO. 5:
5′-AAT GAT ACG GCG ACC ACC GA-3′
SEQ ID NO. 6:
5′-CAA GCA GAA GAC GGC ATA CGA-3′

The results of the inventors positively proved the principle that next generation library for amplicon sequencing can be successfully and rapidly prepared with a single ligation step if the amplicons are generated using 5′-phosphorylated primers, eliminating time-consuming and error-prone multiple enzyme steps that are required in the methods of the art.

1. A method for generating ligation-ready DNA amplicons of a target DNA, comprising
(i) contacting in a polymerase chain reaction (PCR) buffer said target DNA with at least one DNA polymerase, a dNTP mixture, and at least one PCR primer pair consisting of two target specific PCR primers, to obtain a reaction mixture,
(ii) subjecting said reaction mixture to a PCR to generate a plurality of ligation-ready DNA amplicons of said target DNA,
wherein at least one of said target specific PCR primers is 5′-phosphorylated.

2. The method of claim 1, wherein said DNA polymerase has no 3′-5′ exonuclease activity and is a Taq polymerase.

3. The method of claim 1, wherein said DNA polymerase has a 3′-5′ exonuclease activity and is a Pfu or KOD polymerase.

4. The method of claim 1, wherein the plurality of ligation-ready DNA amplicons are configured for ligation with a DNA adaptor molecule, wherein the DNA adapter molecule comprises a nucleotide sequence for annealing an oligonucleotide.

5. The method of claim 1, further comprising
(iii) isolating said plurality of ligation-ready DNA amplicons of said target DNA from said reaction mixture.

6. A method for generating DNA-adaptor-ligated DNA amplicons of a target DNA, comprising
(i) contacting in a polymerase chain reaction (PCR) buffer said target DNA with at least one DNA polymerase, a dNTP mixture, and at least one PCR primer pair consisting of two target specific PCR primers, to obtain a reaction mixture,
(ii) subjecting said reaction mixture to a PCR to generate a plurality of ligation-ready DNA amplicons of said target DNA,
(iii) isolating said plurality of ligation-ready DNA amplicons of said target DNA from said reaction mixture,
(iv) ligating said ligation-ready DNA amplicons of said target DNA to at least one DNA adaptor molecule to generate DNA-adaptor-ligated DNA amplicons of said target DNA,
wherein at least one of said target specific PCR primers is 5′-phosphorylated.

7. The method of claim 6, wherein said DNA polymerase has no 3′-5′ exonuclease activity, has terminal transferase activity and is a Taq polymerase.

8. The method of claim 7, wherein said DNA polymerase has 3′-5′ exonuclease activity and is a Pfu or KOD polymerase.

9. The method of claim 1, wherein said DNA adaptor molecule comprises a sequence for annealing an oligonucleotide, a PCR and/or sequencing primer, or a clonal amplification primer.

10. The method of claim 10, wherein said sequencing primer is a primer for next generation sequencing (NGS).

11. (canceled)

12. (canceled)

13. (canceled)

14. (canceled)

15. A method for generating ligation-ready DNA amplicons of a target DNA, the method comprising amplifying the target DNA using at least one 5′-phosphorylated PCR primer.


SEQUENCE LISTING (6 sequences)

SEQ ID NO: 1 - DNA, 20 bp, Artificial Sequence
  Synthetic construct
cgggtaggcgctctctatgt

SEQ ID NO: 2 - DNA, 23 bp, Artificial Sequence
  Synthetic construct
aagactgacaatcccgtgtaagg

SEQ ID NO: 3 - DNA, 58 bp, Artificial Sequence
  Synthetic construct
aatgatacggcgaccaccgagatctacactctttccctacacgacgctcttccgatct

SEQ ID NO: 4 - DNA, 60 bp, Artificial Sequence
  Synthtic construct
gatcggaagagcggttcagcaggaatgccgagaccgatctcgtatgccgtcttctgcttg

SEQ ID NO: 5 - DNA, 20 bp, Artificial Sequence
  Synthetic construct
aatgatacggcgaccaccga

SEQ ID NO: 6 - DNA, 21 bp, Artificial Sequence
  Synthetic construct
caagcagaagacggcatacga